(19) United States
(12) Patent Application Publication
(10) Pub. No.: US 2026/0098867 A1
(43) Pub. Date: Apr. 9, 2026

(54) METAL NANOPARTICLE, AND PLASMONIC BIOSENSOR COMPRISING THE SAME

(71) Applicant: Korea University Research and Business Foundation, Seoul (KR)

(72) Inventors: Sang Jun SIM, Seoul (KR); Seung Jong BAEK, Seoul (KR)

(73) Assignee: Korea University Research and Business Foundation, Seoul (KR)

(21) Appl. No.: 19/309,845

(22) Filed: Aug. 26, 2025

(30) Foreign Application Priority Data
Aug. 27, 2024 (KR) 10-2024-0115115
Aug. 25, 2025 (KR) 10-2025-0118621

(51) Int. Cl. G01N 33/68; G01N 21/65; G01N 33/543; G01N 33/573

(52) U.S. Cl. CPC G01N 33/6869; G01N 21/658; G01N 33/54346; G01N 33/573; G01N 2333/5415; G01N 2333/91142; G01N 2800/26

(57) ABSTRACT

The present invention relates to metal nanoparticles and a plasmonic biosensor including the same. According to the present invention, it is possible to provide novel metal nanoparticles having significantly improved sensitivity to light, and further, it is possible to provide a plasmonic biosensor capable of detecting a trace amount of a biomarker present in a biological sample with high precision through the metal nanoparticles. In addition, the plasmonic biosensor is capable of detecting a sepsis biomarker with high sensitivity and specificity, and thus may be usefully used in various clinical applications such as sepsis diagnosis, identification of the type of organ dysfunction, prediction of sepsis severity, and post-treatment.

### CROSS REFERENCE TO RELATED APPLICATIONS

This application claims the benefit under 35 USC § 119(a) of Korean Patent Application No. 10-2024-0115115, filed on Aug. 27, 2024 and Korean Patent Application No. 10-2025-0118621, filed on Aug. 25, 2025, the entire disclosure of which is incorporated herein by reference for all purpose.

### TECHNICAL FIELD

The present invention relates to metal nanoparticles and a plasmonic biosensor including the same.

### BACKGROUND ART

Sepsis is a severe infectious disease that causes a systemic inflammatory response in the body and organ dysfunction due to microbial infection, and is caused by various microorganisms, including bacteria, fungi, parasites, and viruses. According to Statistics Korea, sepsis is the 9^{th} leading cause of death in Korea in 2023, and is the second leading cause of death after pneumonia among infectious diseases. According to WHO statistics, 11 million people died from sepsis worldwide in 2020, accounting for about 20% of all deaths. Therefore, sepsis is a disease for which prompt and accurate diagnosis and treatment are crucial.

In the stages of sepsis, infection progresses to sepsis with organ dysfunction, which then progresses to septic shock with hypotensive shock. As each stage progresses, the mortality rate increases rapidly (approximately 10% to 50%), and thus prompt diagnosis and antibiotic administration are essential. Current methods for diagnosing sepsis include a method of quantifying the concentrations of procalcitonin and C-reactive protein to determine the degree of bacterial infection and inflammation, a sequential organ failure assessment (SOFA) scoring method of determining the degree of organ failure by scoring physiological indicators for six organs, and a blood culture method of identifying the causative agent. However, these methods have the disadvantage of being time-consuming or having low accuracy in diagnosing sepsis. In particular, the problem in the medical field is that it is difficult to distinguish between non-infectious organ dysfunction and sepsis-induced organ dysfunction within a significantly short time. That is, both types of organ dysfunction show similar values in the above methods, and in the case of the blood culture method, it takes several days to get the results, and there is a fatal drawback in that false negative results often occur when the causative agent dies inside the body. In addition, antibiotic cocktails are administered for the treatment of sepsis, but it is very important to distinguish the type of organ dysfunction because this treatment is not very effective for non-infectious organ dysfunction and can trigger antibiotic resistance problems. It is also necessary to diagnose the severity of sepsis, as adjusting the amount of antibiotics depending on the severity of sepsis can improve the efficiency of treatment and minimize the side effects of antibiotic resistance. Therefore, to solve these problems, it is necessary to discover markers with excellent clinical significance and develop a platform that enables precise diagnosis with excellent sensitivity and specificity.

Meanwhile, cytokines have recently been attracting attention as markers of sepsis. Cytokines are relatively small proteins (generally about 20 kDa in size) that are secreted from various types of cells and play an important role in the expression and regulation of immune and inflammatory responses while regulating the activation, proliferation, and differentiation of immune cells. Since a major characteristic of sepsis is a cytokine storm in which cytokines are secreted in abnormally large amounts due to an excessive systemic immune response to infection, there is a close relationship between the expression levels of cytokines and sepsis. The expression levels of these cytokines may also be effective in differentiating between non-infectious organ dysfunction and infectious organ dysfunction, as the inflammatory stimuli in these two types of organ dysfunction are different. In the case of non-infectious organ dysfunction, damage-associated molecular patterns (DAMPs) act as inflammatory stimuli, and in the case of infectious organ dysfunction, pathogen-associated molecular patterns (PAMPs) act as inflammatory stimuli, and in the latter case, there is a report that cytokine secretion increases explosively. Therefore, it is expected that analyzing the amount of specific cytokines secreted will be useful not only for distinguishing the type of organ dysfunction but also as an indicator for determining the severity of sepsis.

Meanwhile, metal nanoparticles are receiving much attention in various fields, including materials, communications, environment, energy, and biotechnology, due to their unique optical properties and applicability. In particular, surface-enhanced Raman scattering (SERS)-based nanoplasmonic biosensors using metal nanoparticles are attracting attention as a technology for detecting small amounts of biomarkers. SERS refers to the amplification of Raman scattering between the gaps of noble metal nanoparticles or nanostructures such as gold or silver, and this is controlled by the unique optical properties of the metal nanoparticles due to the localized surface plasmon resonance (LSPR) phenomenon. The LSPR phenomenon refers to a collective oscillation phenomenon of free electrons of metal nanoparticles that occurs at the boundary between the surface of the metal nanoparticles and the surrounding dielectric by the interaction of the free electrons of the metal nanoparticles with light when light is irradiated on the surface of the metal nanoparticles having a size smaller than the wavelength of light. When a target material is present around metal nanoparticles, resonance conditions vary depending on the type and amount of target material, and thus the LSPR phenomenon is attracting attention as a sensing platform for high-sensitivity optical signal analysis. However, existing SERS-based biosensor platforms have limitations in applying them to clinical samples containing diverse molecules, as the SERS spectral characteristics include background noise, making discrimination difficult.

### DISCLOSURE

### Technical Problem

The present invention has been made in order to solve the above-described problems, and the present invention is intended to provide novel metal nanoparticles having significantly improved optical performance due to their precisely controlled particle shape, a plasmonic biosensor including the metal nanoparticles, and a method of detecting a biomarker using the plasmonic biosensor.

### Technical Solution

To solve the above technical problem, the present invention provides a metal nanoparticle having a truncated octahedral structure composed of six (100) facets and eight (111) facets, wherein the edges between four different adjacent (100) facets positioned with respect to any one of the (100) facets are truncated to form quadrangular connecting facets, wherein a concave channel is formed in each of the connecting facets.

According to the present invention, the channel may have a quadrangular groove shape having the edges as the center line. In this case, the channel may have a rectangular groove shape.

According to the present invention, the (100) facets with truncated edges may have a quadrangular shape.

According to the present invention, the (111) facets with truncated edges may have a triangular shape.

According to the present invention, the ratio of the area of the (111) facet with truncated edges to the area of the (100) facet with truncated edges may be 5.3 to 6.5.

According to the present invention, the metal may be any one selected from the group consisting of gold (Au), silver (Ag), copper (Cu), platinum (Pt), and palladium (Pd).

The present invention also provides a plasmonic biosensor including: a substrate; and a metal nanoparticle array in which a plurality of the metal nanoparticles are arranged on the substrate.

According to the present invention, the metal nanoparticle array may be configured so that the plurality of metal nanoparticles are in contact with each other.

According to the present invention, a capture antibody may be bound to the metal nanoparticles.

According to the present invention, the plasmonic biosensor may further include a Raman probe including a gold nanoparticle and a detection antibody bound to the gold nanoparticle.

According to the present invention, the plasmonic biosensor may be used to detect a sepsis biomarker.

In this case, the sepsis biomarker may be a myokine or an adipokine.

In addition, the myokine may be oncostatin M or leukemia inhibitory factor (LIF).

In addition, the adipokine may be visfatin.

The present invention also provides a method of detecting a sepsis biomarker using the plasmonic biosensor.

The features and advantages of the present invention will become more apparent from the following detailed description taken with reference to the accompanying drawings.

The terms or words used in the specifications and claims should not be construed as usual or dictionary definition but should be rather construed with meanings and concepts that are consistent with the technical spirits of the present invention based on the principle that the inventor may properly define the terms to describe his/her invention in the best manner.

### Advantageous Effects

According to the present invention, it is possible to provide novel metal nanoparticles having significantly improved sensitivity to light, and further, it is possible to provide a plasmonic biosensor capable of detecting a trace amount of a biomarker present in a biological sample with high precision through the metal nanoparticles. In addition, the plasmonic biosensor is capable of detecting a sepsis biomarker with high sensitivity and specificity, and thus may be usefully used in various clinical applications such as sepsis diagnosis, identification of the type of organ dysfunction, prediction of sepsis severity, and post-treatment.

### BRIEF DESCRIPTION OF DRAWINGS

FIG. 1 schematically shows a process of producing novel metal nanoparticles (3D nanoparticles with nanotrenches; 3DNT) according to the present invention and a process of manufacturing a diagnostic platform for classification of organ dysfunction and diagnosis of sepsis severity.

FIGS. 2A to 2G show the formation principle of gold nanoparticles according to the present invention and the results of analyzing the structure thereof. Specifically, (A) schematically shows the synthesis of gold nanoparticles (Au 3DNTs) according to the present invention using S7 peptide, (B) is an SEM image of truncated octahedral gold nanoparticles (Au TOh), (C) is an SEM image of Au 3DNTs, (D) shows SEM images of Au 3DNT enlarged in three directions, (E) is an XRD spectrum of Au 3DNT, (F) shows the results of analyzing Au 3DNTs with various dimensions, and (G) is an SEM image of a substrate having Au 3DNTs arranged thereon.

FIGS. 3A to 3D show Lumerical FDTD simulation data of gold nanoparticles Au 3DNTs according to the present invention. Specifically, (A) shows the charge distribution of Au 3DNTs, (B) shows the degree of electric field amplification of a single Au 3DNT, (C) shows the degree of electric field amplification in an array of Au 3DNTs, and (D) shows an SEM image and the degree of electric field amplification when a 15 nm spherical particle probe is bound to an array of Au 3DNTs.

FIGS. 4A to 4D show the results of evaluating the performance of a gold nanoparticle Au 3DNT-based SERS biosensor according to the present invention. Specifically, (A) shows Raman spectra taken at 100 random locations to evaluate reproducibility, (B) shows the results of evaluating specificity, (C) shows the results of evaluating sensitivity for three cytokines (oncostatin M, LIF, and visfatin), and (D) shows the results of linear regression analysis.

FIG. 5 shows the results of evaluating the clinical significance of a gold nanoparticle Au 3DNT-based SERS biosensor according to the present invention. Specifically, it shows the results of analyzing cytokine expression levels for 10 patients in each of five patient groups (healthy control group, non-infectious organ dysfunction group, infection group, sepsis group, and septic shock group): oncostatin M (A), LIF (B), and visfatin (C). The portion marked in red indicates the section with the lowest p value in distinguishing each patient group.

FIG. 6 shows the results of analyzing the accuracy of organ dysfunction classification and sepsis severity assessment through machine learning analysis based on the SVM algorithm. Specifically, (A) is a heatmap graph showing the accuracy of diagnosis based on the combination of cytokine biomarkers, (B) is a confusion matrix graph showing the result of classification performed based on the combination of three cytokines, and (C) is a confusion matrix graph showing the result of classification performed by integrating three cytokines with SOFA scores.

FIG. 7 is a detailed schematic diagram of a process of manufacturing a diagnostic platform for identification of organ dysfunction and diagnosis of sepsis severity.

FIG. 8 is an SEM image of gold nanoparticles produced without S7 peptide.

FIG. 9 shows graphs showing reproducibility shown by measuring signals at 30 random points of a gold nanoparticle Au-based 3DNT SERS biosensor according to the present invention at a target cytokine concentration of 100 pM.

### MODE FOR INVENTION

Unless otherwise defined, all technical and scientific terms used in the present specification have the same meanings as commonly understood by those skilled in the art to which the present invention pertains. In general, the nomenclature used in the present specification is well known and commonly used in the art.

In the present invention, the term “nano-plasmonic biosensor” refers to a biosensor that is capable of measuring plasmon, wherein the plasmon means a quantum of an oscillation of electron or hole density, that is, a quantum of plasma oscillation, and that includes a measuring unit for measuring plasmon which is a quasiparticle associated with a collection of oscillations of free electrons in a metal.

In the present invention, the term “substrate” refers to a plate to which the metal nanoparticles may be fixed to allow observation under a microscope. The substrate may be, for example, a silicon substrate or a glass slide, without being limited thereto.

In order to amplify SERS, it is necessary to precisely control the shape of particles to form gaps within gold nanoparticles or between gold nanoparticles. For this purpose, in the present invention, a peptide was used as a capping agent for nanoparticle synthesis. In the case of peptides, a wide variety of sequences may be generated by assembling 20 amino acids, and the shape of nanoparticles may be pre-designed by generating peptides of a specific sequence. In addition, because the peptides are biomolecules, they are more advantageous for application as biosensors in the future. The S7 peptide (sequence: Ac-Ser-Ser-Phe-Pro-Gln-Pro-Asn-CONH₂) is known to have high binding affinity and specificity for the {111} facets of metals (gold, silver, platinum, copper, etc.) having a face-centered cubic (FCC) structure. This has been proven through molecular dynamics simulations because the phenyl ring, which is the phenylalanine residue, the third amino acid present in the S7 peptide, epitaxially matches the hexagonal atomic arrangement of the {111} facet of the FCC metal.

Under this technical background, the present invention is intended to provide novel metal nanoparticles having remarkably improved optical performance due to their precisely controlled particle shape, a plasmonic biosensor including the metal nanoparticles, and a method of detecting a biomarker using the plasmonic biosensor.

To this end, the present invention provides a metal nanoparticle having a truncated octahedral structure composed of six (100) facets and eight (111) facets, wherein the edges between four different adjacent (100) facets positioned with respect to any one of the (100) facets are truncated to form quadrangular connecting facets, wherein a concave channel is formed in each of the connecting facets.

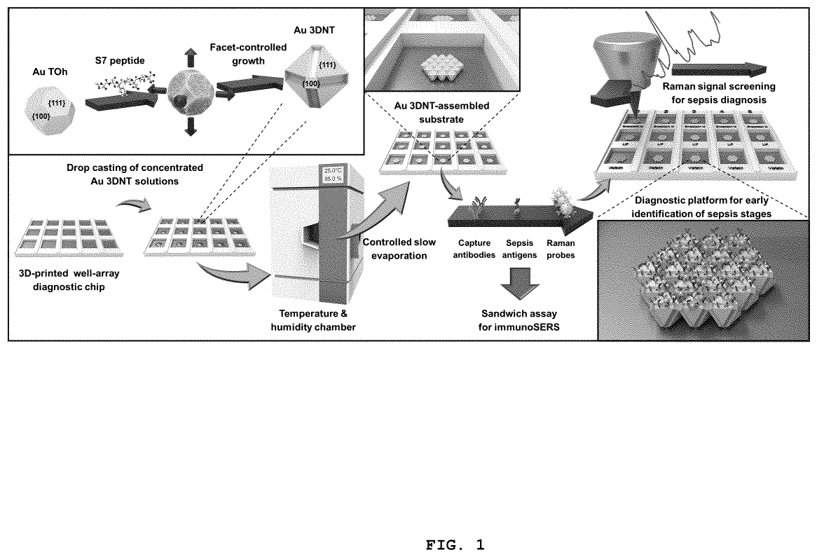

Specifically, in the present invention, using the characteristic of a capping agent that selectively adsorbs only to a specific facet and reduces the activation energy of the facet, thus controlling growth, truncated-octahedral gold nanoparticles (Au TOh) with a mixture of {111} and {100} facets were first synthesized, selected as starting particles, incubated with the S7 peptide, and then grown. As shown in FIG. 1, novel gold nanoparticles (Au 3D nanoparticles with nanotrenches; 3DNT) were synthesized in which the area of the selectively capped {111} facet is increased and the area of the non-capped {100} facet is reduced, thereby forming 12 trench-like structures while controlling the facets.

As can be seen from the results of the Examples below, the gold nanoparticles (Au 3DNTs) according to the present invention have the characteristics of Au TOh that are advantageous for arrangement, and thus are advantageous for forming a uniform particle layer. In addition, the nanoparticle has elongated trench structures therein, and thus a “hot spot” region where the electromagnetic field between the particles and within the particles is amplified is over-formed, enabling ultrasensitive diagnosis of sepsis-specific biomarkers, which is extremely advantageous not only for distinguishing the type of organ dysfunction but also for precisely determining the severity of sepsis.

In the present invention, it is preferable that the channel has a quadrangular groove shape having the edges as the center line, and it is more preferable that the channel has a rectangular groove shape.

In the present invention, the (100) facets with truncated edges in the metal nanoparticle preferably have a quadrangular shape.

In the present invention, the (111) facets with truncated edges in the metal nanoparticle preferably has a triangular shape.

In this case, the ratio of the area of the (111) facet with truncated edges to the area of the (100) facet with truncated edges in the metal nanoparticles may be 5.3 to 6.5.

In the present invention, the metal may be any one selected from the group consisting of gold (Au), silver (Ag), copper (Cu), platinum (Pt), and palladium (Pd), and preferably may be gold (Au).

The present invention also provides a plasmonic biosensor including: a substrate; and a metal nanoparticle array in which a plurality of the metal nanoparticles are arranged on the substrate.

In the present invention, the metal nanoparticle array may be configured so that the plurality of metal nanoparticles are in contact with each other.

In the present invention, a capture antibody is preferably bound to the metal nanoparticles.

In the present invention, the plasmonic biosensor preferably further includes a Raman probe including a gold nanoparticle and a detection antibody bound to the gold nanoparticle.

In the present invention, the plasmonic biosensor may be used to detect a sepsis biomarker, as can be seen from the results of the Examples below.

In this case, the sepsis biomarker may be a myokine or an adipokine. More specifically, as can be seen from the results of the Examples below, the sepsis biomarker is preferably oncostatin M or leukemia inhibitory factor (LIF) belonging to the interleukin-6 group that regulates inflammatory response among myokines, or visfatin among adipokines.

The present invention also provides a method of detecting a sepsis biomarker using the plasmonic biosensor.

### Examples

Hereinafter, the present invention will be described in more detail through examples. These examples are only to illustrate the present invention, and it will be apparent to those skilled in the art that the scope of the present invention is not construed as being limited by these examples. Thus, the substantial scope of the present invention will be defined by the appended claims and equivalents thereto.

### Experimental Methods

### Synthesis of Au Truncated-Octahedral Nanoparticles (Au TOh)

To produce seed nanoparticles, 7 ml of 100 mM cetrimonium bromide (CTAB) solution, 87.5 μl of 20 mM HAuCl₄ solution, and 600 μl of 10 mM NaBH₄ solution stored at 4° C. for 15 minutes were sequentially added to a 20 ml vial containing a magnetic bar, and the mixture was incubated at 30° C. for 3 hours while maintaining the rotation speed at 700 rpm. Then, for primary growth, 36.3 ml of 16 mM CTAB solution, 75 μl of 20 mM HAuCl₄ solution, 1.161 ml of 38.8 mM ascorbic acid (AA) solution, and 450 μl of the seed solution diluted 100-fold in distilled water (DI) were sequentially added to a 50 ml conical tube, and the mixture was incubated at 30° C. for 12 hours. Finally, for secondary growth, 3 ml of 2 mM HAuCl₄ solution, 4.644 ml of 14 mM AA solution, and 12 ml of the primary growth solution were sequentially added to 12 ml of 50 mM CTAB solution preheated in an oven at 70° C., and the mixture was incubated at room temperature for 10 minutes. Then, the process of centrifugation at 6,510 g for 10 minutes, supernatant removal, and dilution in DI water was repeated twice.

### Synthesis of Au 3DNTs

After diluting Au TOh to an optical density (OD) of 1, 650 μl of the Au TOh solution with an OD of 1 and 350 μl of 1 mg/ml S7 peptide solution were mixed and incubated for 1 hour. Then, 8.680 ml of DI water, 120 μl of 100 mM CTAB solution, 100 μl of 1 mM HAuCl₄ solution, 100 μl of 15 mM AA solution, and 1 ml of the Au TOh-S7 peptide solution were sequentially added to a 50 ml conical tube, and the mixture was incubated at 40° C. for 30 minutes. Then, the process of centrifugation at 5,000 g for 10 minutes, supernatant removal, and dilution in 1 mM CTAB solution was repeated twice.

### Fabrication of Au 3DNT Array Substrate

Au 3DNT was centrifuged under the same conditions (5,000 g for 10 minutes) and concentrated to an OD of 200. A 7 mm×7 mm silicon substrate was ultrasonically washed in 99.9% ethanol and DI water for 15 minutes each, dried with nitrogen gas, and combined with the well of a diagnostic chip fabricated using a 3D printer. Next, 1.2 μl of the Au 3DNT solution with an OD of 200 was dropped onto the silicon substrate and dried for 48 hours in a constant temperature and humidity chamber set at 25° C. and 95% relative humidity.

### Evaluation of SERS Activity of Au 3DNT Array Substrate

5 μl of a solution of 10 μM MGITC in ethanol was dropped onto the Au 3DNT array substrate and dried at room temperature. Then, 100 random locations on the substrate were irradiated with a 785 nm laser (optical power: 10.67 mW) at 32% intensity for 0.5 seconds, and a Raman spectrum was measured to determine the signal uniformity.

### Functionalization of Au 3DNT Array Substrate

The Au 3DNT array substrate was immersed in a 10 mM 11-MUA solution and incubated for 12 hours, and then 5 μl of 500 mM NHS/EDC (in 10 mM MES buffer) was dropped onto the substrate which was then incubated for 10 minutes, followed by washing with DI water, thereby functionalizing the substrate. 2.5 μl of a 1 mg/ml capture antibody solution was dropped onto the NHS/EDC-activated Au 3DNT array substrate which was then incubated for 30 minutes. Thereafter, to prevent nonspecific binding, 2.5 μl of 1% bovine serum albumin (in 0.1×PBS buffer) was additionally dropped onto the substrate which was then incubated for 30 minutes, followed by washing with 0.1×PBS buffer.

### Bonding to Au 3DNT Array Substrate

An SERS probe was prepared by binding MGITC and detection antibody to spherical gold nanoparticles. 10 μl of 10 μM MGITC solution was added to 1 ml of 15-nm spherical gold nanoparticle solution (OD 1), and the mixture was incubated for 1 hour, and centrifuged at 15,000 rpm at 4° C. for 80 minutes. The supernatant was removed, and the remaining material was diluted again in DI water. Next, 2 μl of 10 mM 11-MUA solution was added thereto, and the mixture was incubated for 12 hours, and centrifuged at 15,000 rpm at 4° C. for 80 minutes. The supernatant was removed, and the remaining material was diluted again in DI water. 1.25 μl of 50 mM NHS/EDC solution was added to 500 μl of the 15-nm spherical gold nanoparticle solution treated with MGITC and 11-MUA, and the mixture was incubated for 15 minutes at room temperature. After 15 minutes, 1.25 μl of 1 mg/ml detection antibody solution was added to the resulting solution which was then incubated. After another 15 minutes, to prevent nonspecific binding, 1.25 μl of 1.0 mM ethanolamine solution was added thereto and the mixture was incubated for another minutes. After completion of the incubation, the supernatant was removed by centrifugation at 15,000 rpm at 4° C. for 60 minutes, and the remaining material was diluted again in 0.1×PBS buffer.

### Clinical Sample Collection

Clinical samples were provided by Korea University Ansan Hospital (IRB #2023AS0361). For the serum used in the experiment, the blood of the volunteers was placed in serum separation tubes, centrifuged at 5,000 rpm for 30 minutes, and the supernatants were carefully transferred to cryogenic tubes and stored at −80° C.

### SERS Immunoassay for Detection of Sepsis

For precise analysis, the patients' serum samples were diluted 10-fold in 0.1×PBS buffer, and 5 μl of the dilution was dropped onto the capture antibody-treated Au 3DNT array substrate, which was then incubated for 30 minutes and washed with 0.1×PBS buffer. Thereafter, 5 μl of the SERS probe solution was dropped to induce sandwich binding for 30 minutes, and then washed with 0.1×PBS buffer in the same manner. The measurement of SERS signals was done 40 times by a 785 nm laser (optical power: 10.67 mW) at 32% intensity for 0.5 seconds each, and the signal intensity at 1170 cm-1, which is a characteristic peak of MGITC, was quantified to analyze the performance of the substrate and expression levels.

### Numerical Calculation

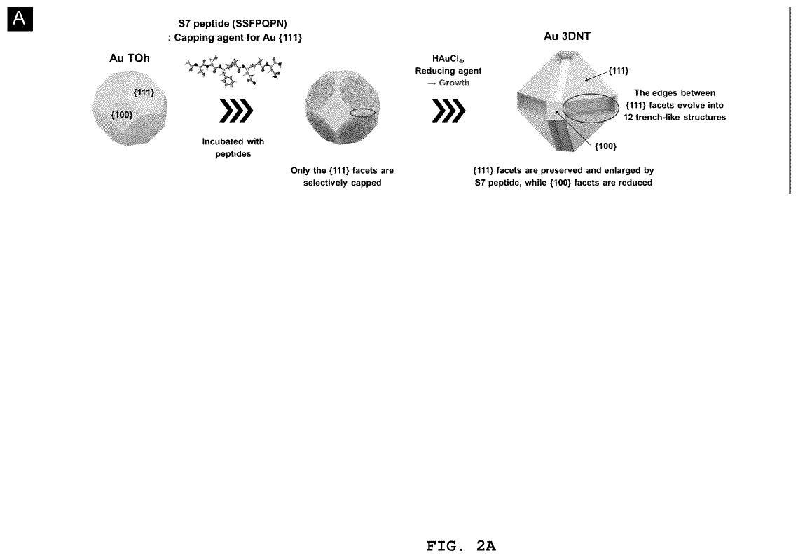

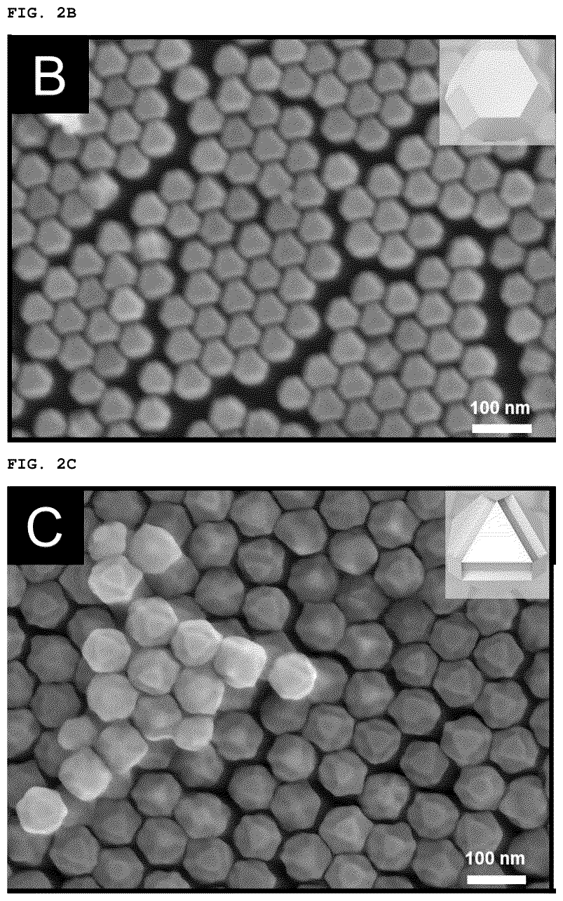

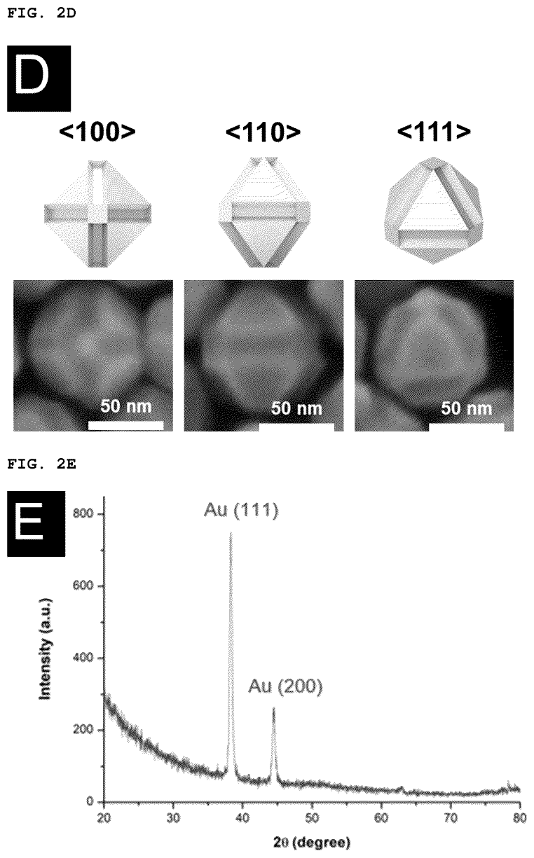

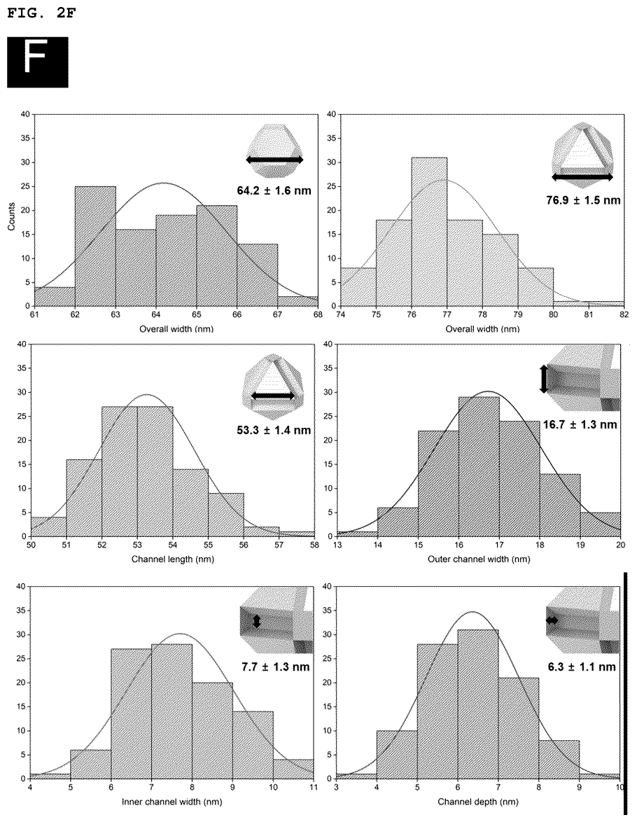

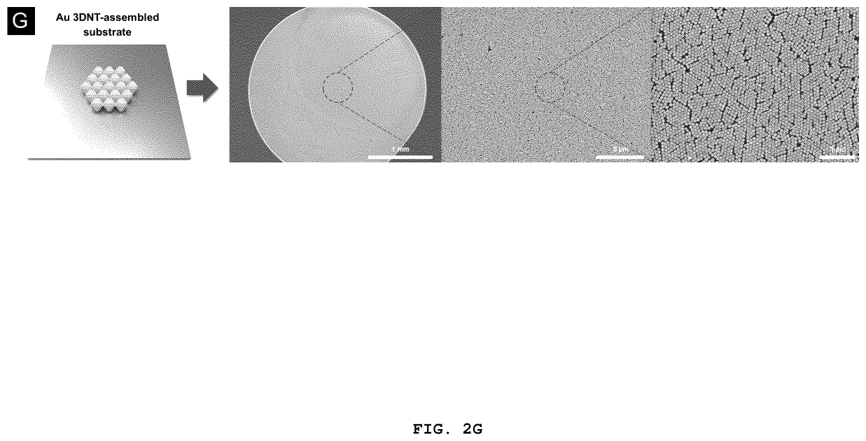

The electric field profiles for Au 3DNT and Au 3DNT-based platforms were calculated using Lumerical FDTD Solutions software (Lumerical Inc.). The particle size data used in the simulations were the average values shown in FIG. 2F, and the light wavelength was fixed at 785 nm, which was used in the actual SERS measurements. Additionally, the mesh size was set to 0.5 nm.

### Machine Learning-Based Classifications

Clinical data were trained using the support vector machine (SVM) algorithm. Cross-validation was used, with 90% of the entire dataset used for training and 10% for validation, alternating between datasets. Additionally, confusion matrices, accuracy, precision, sensitivity, and specificity were calculated to demonstrate the performance of the classification model.

### Results and Discussion

### Preparation and Characterization of Gold Nanoparticle Au 3DNT-Based SERS Biosensor

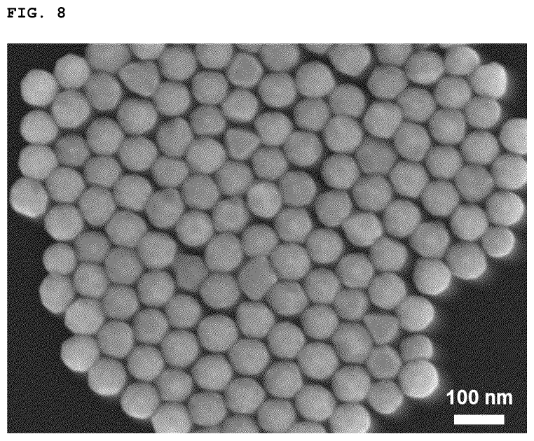

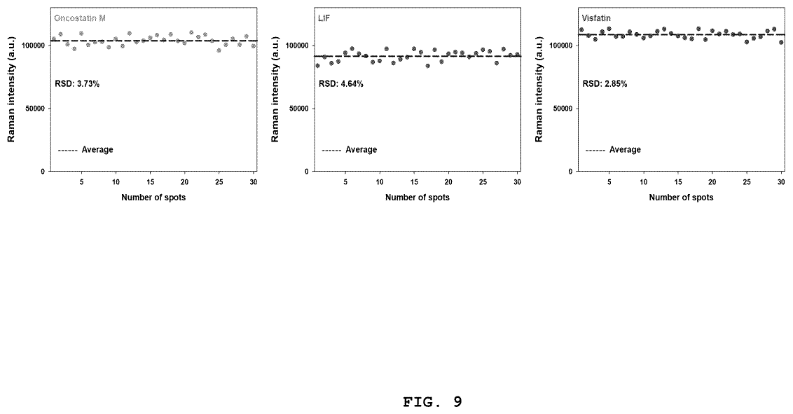

In order to detect sepsis target cytokines with high sensitivity and specificity, a SERS substrate with high-density hot spots must be fabricated. The SERS substrate fabrication methods can be divided into a top-down method that forms large-area nanostructures through methods such as deposition and etching, and a bottom-up method that arranges nanoparticles on a substrate. In the case of the top-down method, there is an advantage in that high uniformity of the substrate may be secured due to process advantages, but there is a disadvantage in that the sensitivity is low due to the difficulty in fine patterning. On the other hand, the bottom-up method has high sensitivity due to the microscopic gaps between the nanoparticles themselves and the nanoparticle arrays, but has the disadvantage of low uniformity due to the difficulty in uniform synthesis and arrangement. To overcome the disadvantages of such nanoparticle-based fabrication methods, (1) the nanoparticles themselves must have high uniformity and (2) they must have the property of being well arranged. Au TOh is a nanoparticle composed of eight {111} facets and six {100} facets, and is known to have very high alignment efficiency due to the van der Waals force between the flat {100} facets. For this reason, Au TOh was selected as the starting particle for peptide application. The selected peptide is a peptide named S7, which consists of a seven-amino acid sequence (Ac-Ser-Ser-Phe-Pro-Gln-Pro-Asn-CONH₂). The phenyl ring of phenylalanine present in the sequence epitaxially matches the hexagonal atomic arrangement of the {111} facet of a metal having an fcc structure, and thus has the characteristic of being stabilized by taking a lying down shape and specifically protecting the {111} facet while being adsorbed. On the other hand, the phenyl ring is structurally inconsistent with the {100} facet with a square atomic arrangement, and thus becomes unstable by taking a standing shape and detaches from the facet. By applying the S7 peptide to the growth of Au TOh based on this principle, Au 3DNT was synthesized by specifically adsorbing the peptide only onto the {111} facets to cap them and performing growth to reduce the {100} facets and enlarge the {111} facets while allowing the edges between the {111} facets to evolve into 12 trench-like structures (FIG. 2A). Next, the synthesized nanoparticles were analyzed using field-emission scanning electron microscopy (FE-SEM). It was confirmed that both the starting particles, Au TOh (FIG. 2B), and the final particles, Au 3DNT (FIG. 2C), were obtained with high uniformity. To justify the shape of the Au 3DNT, enlarged images in the <100>, <110>, and <111> directions were attached for verification (FIG. 2D). In addition, XRD analysis confirmed that the particle were composed only of {111} and {100} facets as claimed (FIG. 2E). Additionally, numerical analysis was performed on 100 individual particles using Image J software to measure the size distributions for the overall width of Au TOh (64±1.6 nm), the overall width of Au 3DNT (76.9±1.5 nm), the trench length (53.3±1.4 nm), the outer trench width (16.7±1.3 nm), the inner trench width (7.7±1.3 nm), and the trench depth (6.3±1.1 nm) (FIG. 2F). Based on these numerical values, the ratios of {111}/{100} facets of Au TOh and Au 3DNT were calculated and compared, and as a result, {111}/{100} increased by approximately 1.70 times from 3.46 to 5.89. Finally, a small amount of a concentrated solution of Au 3DNT was dropped onto a silicon wafer and slowly dried for 48 hours in a constant temperature and humidity chamber set to 25° C. and a high relative humidity of 95% to minimize the coffee ring effect and form a uniformly arranged particle layer due to the strong cohesion between the particles. This was verified through SEM images at various magnifications (FIG. 2G). On the other hand, as a result of conducting a control experiment without the peptide, it could be observed that highly non-uniform and spherical particles with a mixture of multiple facets were obtained, verifying the influence of the S7 peptide in controlling the facets of the particles (FIG. 8).

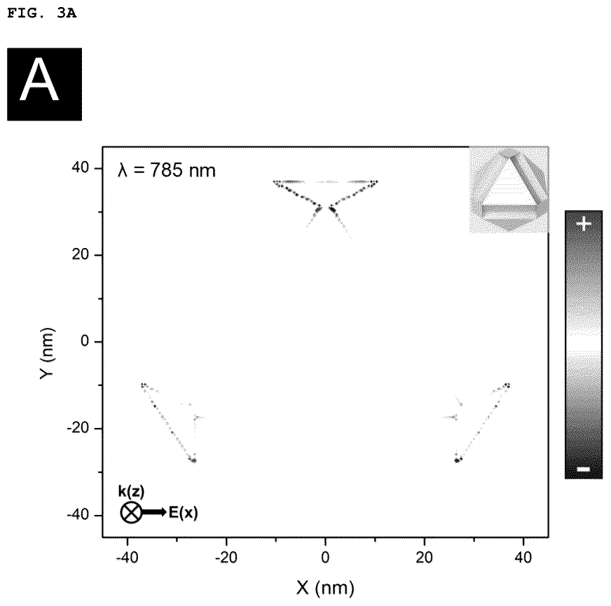

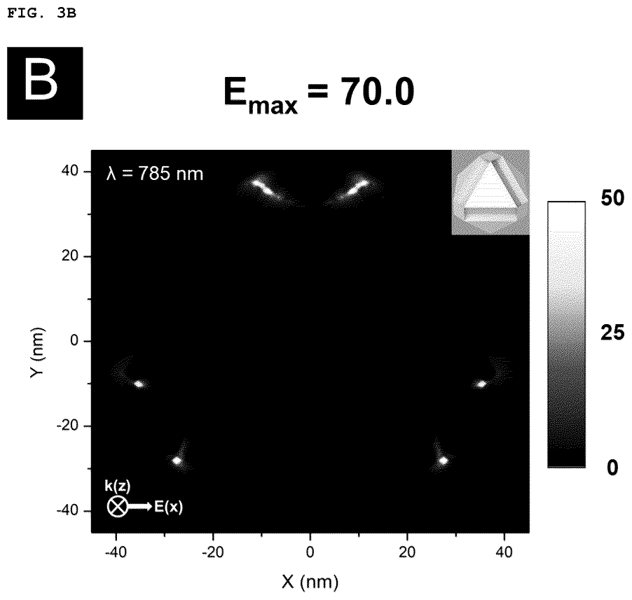

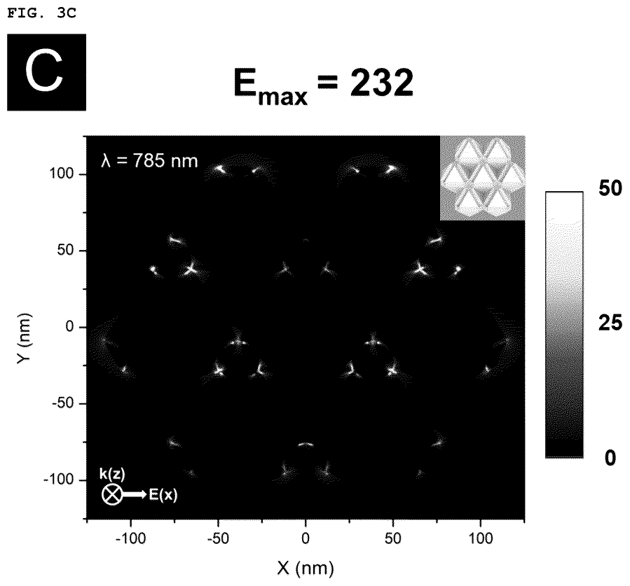

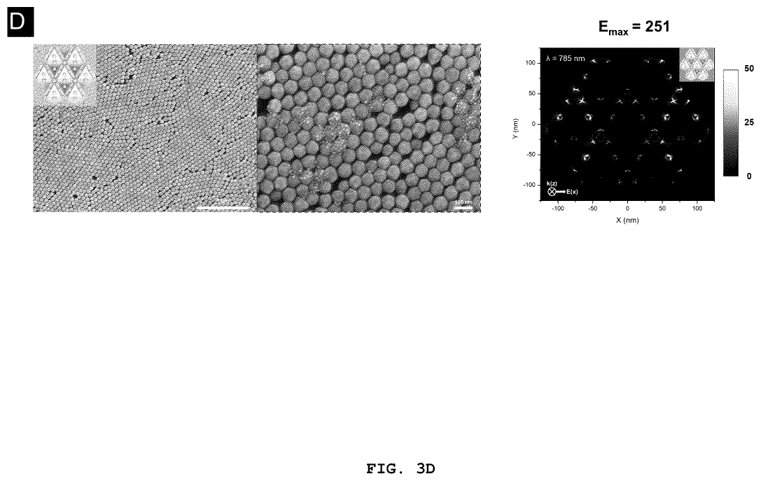

Next, luminal FDTD (finite-difference time-domain) simulations were performed on the synthesized particles. All simulations were performed for light with a wavelength of 785 nm, which is the wavelength at which Raman analysis was performed. First, as a result of performing a charge density distribution simulation on Au 3DNT, it was confirmed that a symmetrical charge distribution was obtained (FIG. 3A). Next, the present inventors performed a simulation of the electric field distribution when a 15-nm spherical probe was bound to a single Au 3DNT particle, an array of the particles, and a particle layer. The simulation results showed that, in the single particle, the electric field amplification occurred mainly at the short edge of the trench structure (FIG. 3B), and in the array of the particles, a large amplification was obtained in portions where the particles gathered 3-fold (FIG. 3C). Finally, a sandwich assay was performed on the particle layer to determine how much signal amplification was achieved when a 15 nm spherical probe was introduced. It was confirmed that the strongest amplification occurred when the spherical probe was positioned within the trench of an individual particle. The Emax values were 70.0, 232, and 251, respectively, and increased by 3.31 times and 1.08 times as each step progressed.

### Evaluation of Analytical Performance of Gold Nanoparticle Au 3DNT-Based SERS Biosensor

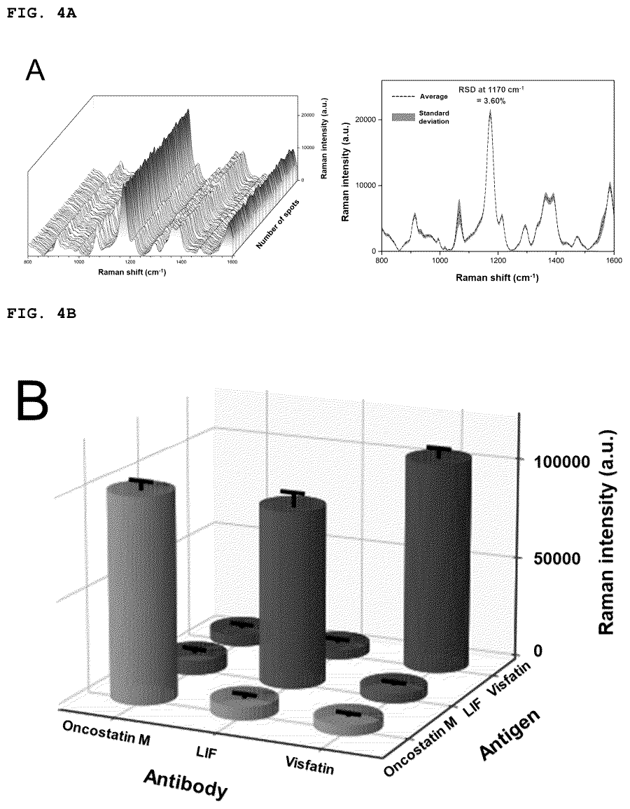

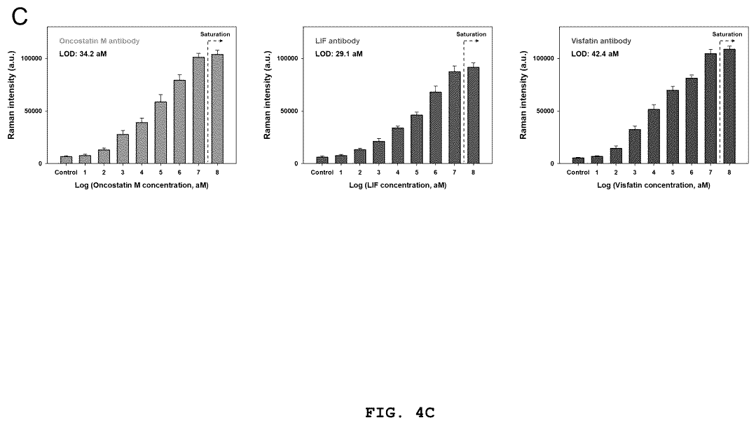

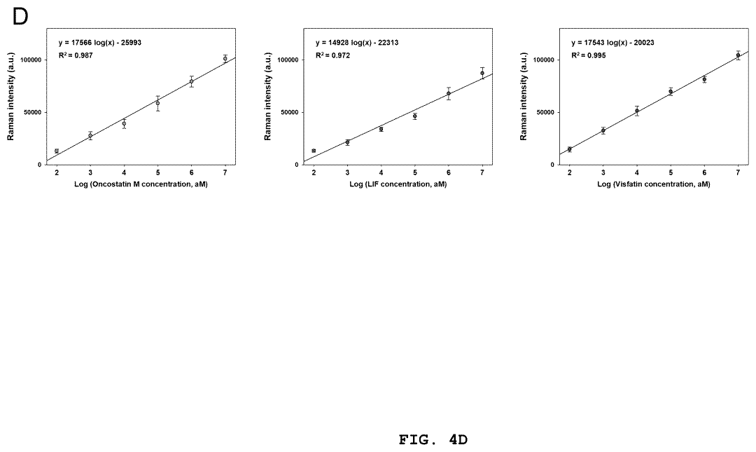

Next, the performance of the Au 3DNT-based SERS biosensor was measured. High reproducibility is essential for the use of SERS substrates in clinical settings. To quantitatively confirm this, 5 μl of the Raman marker MGITC (malachite green isothiocyanate) at a concentration of 10 μM was dropped onto a substrate on which Au 3DNTs were uniformly arranged, dried, and then treated, and the Raman intensity was measured. As a result of displaying the Raman spectrum by measuring signals from 100 random locations, it was confirmed that the relative standard deviation at 1,170 cm-1, where the most characteristic peak of MGITC occurs, was 3.60%, which is an extremely low value of less than 5%, indicating that the biosensor according to the present invention has high reproducibility (FIG. 4A).

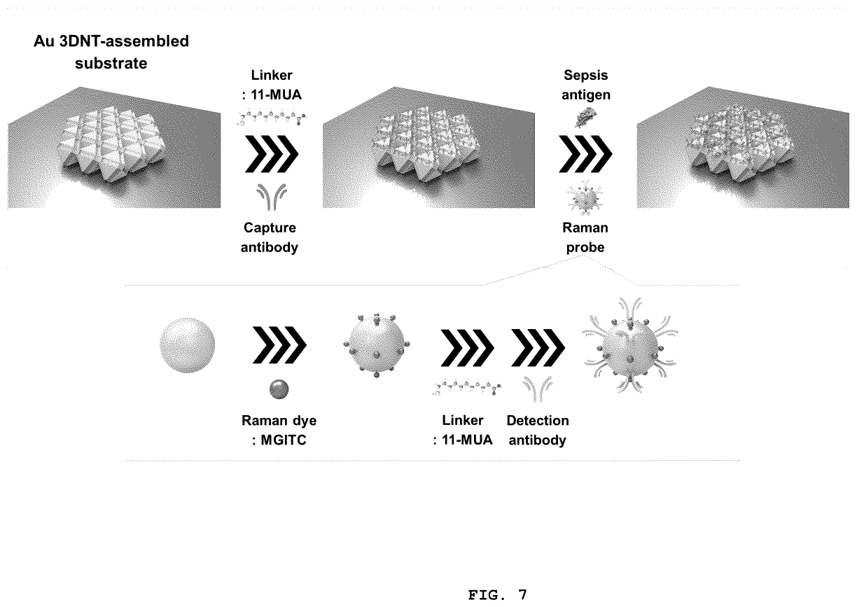

In addition, it is important to verify specificity because there are a wide variety of molecules in actual blood, which act as signal interference factors. In order to check the cross-reactivity of three cytokine markers, the Au 3DNT-based substrate was treated with 11-MUA (11-mercaptoundecanoic acid) as a linker to expose carboxyl groups on the surface of the substrate, and then capture antibodies for each cytokine were bound through the N-hydroxysuccinimide (NHS)-1-ethyl-3-diaminopropyl carbodiimide (EDC) reaction. The substrate was additionally treated with BSA (bovine serum albumin) to prevent nonspecific binding. Thereafter, the substrate was treated with a serum sample (10-fold diluted in 0.1×PBS buffer) containing each cytokine marker and treated with a Raman probe for the target cytokine (FIG. 7). Next, as a result of measuring the Raman signal of each substrate, it was confirmed that high Raman intensity was obtained only for the target cytokine, and very low intensity was obtained for other cytokines (FIG. 4B). This result was obtained even though the concentration of the target cytokine was 10 pM and the concentration of other cytokines was 1 nM, which is 100 times higher. Thereby, it was confirmed that the biosensor according to the present invention has very high specificity. Additionally, through a recovery test in a serum sample, a recovery rate ranging from 95.9% to 104% was confirmed, indicating that the Au 3DNT array substrate according to the present invention can function as a reliable sepsis diagnostic tool (Table 1).

TABLE 1

| Samples | Oncostatin M | LIF | Visfatin |
|---|---|---|---|
| Added (fM) | 10.0 | 10.0 | 10.0 |
| Found (fM) | 9.59 | 10.1 | 10.4 |
| RSD (%) | 5.26 | 5.37 | 3.83 |
| Recovery (%) | 95.9 | 101 | 104 |

Furthermore, sensitivity analysis was performed to verify whether the Au 3DNT array substrate can accurately and precisely distinguish and diagnose sepsis by quantifying the expression level of each cytokine. Because cytokines have lower molecular weights and lower blood concentrations than general proteins, high sensitivity is required. Raman signal intensities for various concentrations of target cytokines were measured, and the analyzable concentration range and detection limit were determined. Raman signal intensities were measured while increasing the concentration by 10-fold from 10 aM to 100 pM, and it was confirmed that the signals for all three cytokines were saturated from 100 pM (FIG. 4C). Additionally, when signals were measured at 30 random locations on the substrate at a concentration of 100 pM, which is a concentration at which the signals are saturated, it was confirmed that the relative standard deviations for all three cytokines showed extremely low values of less than 5%, indicating that the biosensor according to the present invention has high reproducibility even in a state in which a Raman probe is bound thereto (FIG. 9). To determine the detection limit, linear regression was performed over the concentration range from 100 aM to 10 pM, excluding concentrations where the signal overlapped with the control experiment or the signal was saturated, and it was confirmed that the biosensor according to the present invention had an extremely low detection limit of several tens of aM for all three cytokines (FIG. 4D). The specific linear regression equation for each cytokine is graphically shown in FIG. 4D, and the detection limit was calculated as 3.3×5/m (5 is the standard deviation of the blank, and m is the slope of the curve). Thereby, it was confirmed that the Au 3DNT-based SERS biosensor according to the present invention, which has a very low detection limit, can achieve an amplified signal even for a slight difference in the cytokine expression level for each sepsis patient group, and thus is very advantageous for differential diagnosis.

Meanwhile, recent studies have emphasized the need for tailored treatment strategies based on the severity of sepsis, which often begins with infection. Furthermore, distinguishing between non-infectious organ dysfunction and sepsis-induced organ dysfunction is particularly crucial in treatment strategies, because, in the case of non-infectious organ dysfunction, antibiotics may not be effective in alleviating symptoms and can even lead to the development of antibiotic resistance which is an adverse effect. However, the SOFA score, which is currently the most widely used in the diagnosis of sepsis, is a score that quantifies the degree of organ dysfunction. Although the SOFA score is somewhat effective in diagnosing the severity of sepsis, it has a major drawback in that it cannot distinguish between non-infectious organ dysfunction and sepsis-induced organ dysfunction. Therefore, clearly distinguishing between five patient groups (healthy controls (HC), non-infectious organ dysfunction, infection, sepsis, and septic shock) is essential for appropriate antibiotic use and optimal clinical treatment.

To confirm whether differential diagnosis of the above five patient groups is clinically possible, serum samples from a total of 50 patients (10 patients for each patient group) were prepared (specific information for each patient is shown in Table 2 below). A diagnostic chip including a total of 15 wells was easily mass-produced using a 3D printer. A silicon substrate on which Au 3DNTs were arranged was bound to each vertical row, and then treated with capture antibodies for three cytokine markers: oncostatin M, LIF, and visfatin. Thereafter, to minimize the signal interference factors present in serum, 5 μl of serum diluted 10-fold in 0.1×PBS buffer was added to each well which was then incubated, followed by washing. Then, 5 μl of a Raman probe including MGITC and detection antibodies for each cytokine, bound to 15-nm spherical gold nanoparticles, was added to each well which was then incubated and washed, thereby completing treatment of the substrate for SERS measurement. Thereafter, the expression levels of the three cytokines were measured by quantifying the Raman signal intensity at 1,170 cm⁻¹, at which the most characteristic peak of MGITC occurs, and multiple cytokine analysis of five patients was quickly performed on one diagnostic chip through screening.

TABLE 2

| Patient | | Age | Infection | SOFA | Diagnostic |
|---|---|---|---|---|---|
| No. | Gender | (years) | status | score | results |
| 1 | M | 33 | − | ≤1 | HC |
| 2 | F | 28 | − | ≤1 | HC |
| 3 | F | 32 | − | ≤1 | HC |
| 4 | F | 32 | − | ≤1 | HC |
| 5 | F | 32 | − | ≤1 | HC |
| 6 | F | 35 | − | ≤1 | HC |
| 7 | F | 31 | − | ≤1 | HC |
| 8 | M | 31 | − | ≤1 | HC |
| 9 | F | 31 | − | ≤1 | HC |
| 10 | F | 30 | − | ≤1 | HC |
| 11 | M | 61 | − | 6 | Organ |
| | | | | | dysfunction |
| 12 | M | 64 | − | 13 | Organ |
| | | | | | dysfunction |
| 13 | M | 58 | − | 4 | Organ |
| | | | | | dysfunction |
| 14 | M | 63 | − | 15 | Organ |
| | | | | | dysfunction |
| 15 | M | 45 | − | 14 | Organ |
| | | | | | dysfunction |
| 16 | M | 47 | − | 7 | Organ |
| | | | | | dysfunction |
| 17 | F | 34 | − | 6 | Organ |
| | | | | | dysfunction |
| 18 | F | 75 | − | 6 | Organ |
| | | | | | dysfunction |
| 19 | M | 76 | − | 7 | Organ |
| | | | | | dysfunction |
| 20 | M | 54 | − | 3 | Organ |
| | | | | | dysfunction |
| 21 | M | 76 | + | 3 | Infection |
| 22 | F | 78 | + | 5 | Infection |
| 23 | M | 94 | + | 4 | Infection |
| 24 | F | 77 | + | 1 | Infection |
| 25 | M | 44 | + | 5 | Infection |
| 26 | F | 89 | + | 3 | Infection |
| 27 | F | 40 | + | 2 | Infection |
| 28 | F | 65 | + | 2 | Infection |
| 29 | M | 60 | + | 5 | Infection |
| 30 | M | 67 | + | 8 | Infection |
| 31 | M | 89 | + | 11 | Sepsis |
| 32 | F | 83 | + | 7 | Sepsis |
| 33 | M | 76 | + | 6 | Sepsis |
| 34 | M | 84 | + | 7 | Sepsis |
| 35 | M | 53 | + | 8 | Sepsis |
| 36 | M | 74 | + | 8 | Sepsis |
| 37 | F | 84 | + | 7 | Sepsis |
| 38 | F | 87 | + | 4 | Sepsis |
| 39 | M | 76 | + | 5 | Sepsis |
| 40 | F | 89 | + | 8 | Sepsis |
| 41 | M | 73 | + | 7 | Septic shock |
| 42 | M | 68 | + | 5 | Septic shock |
| 43 | M | 79 | + | 12 | Septic shock |
| 44 | M | 76 | + | 14 | Septic shock |
| 45 | M | 84 | + | 15 | Septic shock |
| 46 | M | 84 | + | 13 | Septic shock |
| 47 | F | 81 | + | 11 | Septic shock |
| 48 | M | 91 | + | 8 | Septic shock |
| 49 | M | 82 | + | 6 | Septic shock |
| 50 | M | 84 | + | 17 | Septic shock |

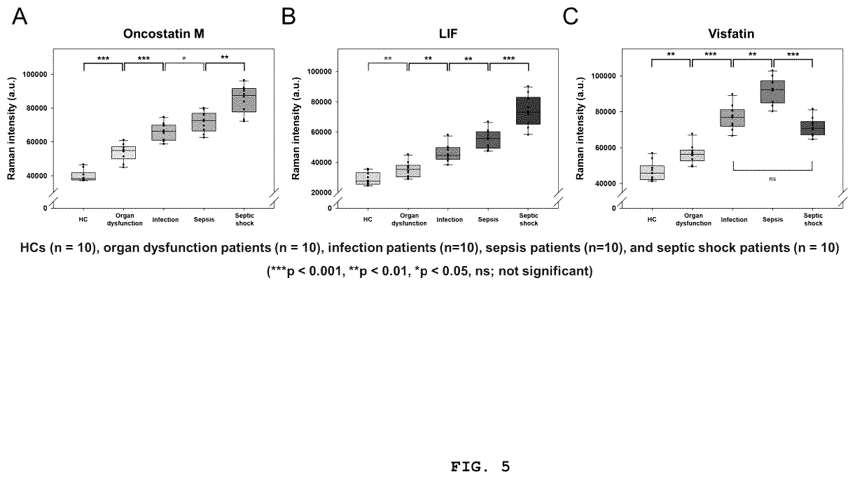

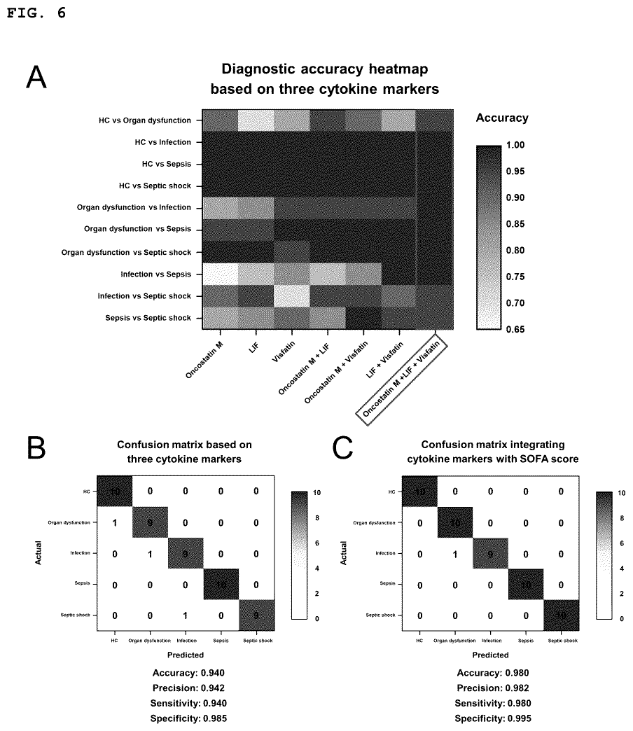

As a result of the analysis, the expression levels of oncostatin M and LIF, which are IL-6 myokines involved in inflammatory response, tended to increase from the healthy control group to non-infectious organ dysfunction, infection, sepsis, and septic shock, and there was a slight difference in the clinical efficacy indicated by the increase amount and p value for each patient group (FIGS. 5A and 5B). This is believed to be because the degree of inflammatory response was lowest in the healthy control group, and was lower in non-infectious organ dysfunction represented by DAMP than in infection represented by PAMP. Specifically, in the case of oncostatin M, it was most difficult to distinguish between infection and sepsis, and in the case of LIF, it was most difficult to distinguish between the healthy control group and non-infectious organ dysfunction. On the other hand, in the case of visfatin, an adipokine, a specific pattern occurred in which the expression level increased rapidly from the healthy control group to sepsis, but the expression level decreased rather than increased in septic shock (FIG. 5C). This is believed to be because visfatin acts as a precursor of IL-6, an inflammatory cytokine, and thus in the early stages of infection, the concentration of visfatin rapidly increases, and in septic shock which is accompanied by hypotension, the concentration of visfatin that is also used in metabolic processes by mimicking insulin decreases. Therefore, in the case of visfatin, it was most difficult to distinguish between infection, where the expression level increases, and septic shock, where the expression level decreases. In order to statistically obtain the p value for the expression level of each cytokine in the five patient groups, the Mann-Whitney U test was performed. The p value was calculated for each patient group, and the results were graphically expressed. In this case, the part marked in red means the section having the lowest p value in the distinction of the patient groups, and the part not marked separately means the case where the p value is less than 0.001.

Next, Raman signal intensity-based expression data for the five patient groups were trained through machine learning to dramatically increase diagnostic accuracy. The support vector machine (SVM) was selected as the machine learning-based analysis model for classification. Cross-validation, which is advantageous for training models with small amounts of data, was selected as the model training method. After training the model, the diagnostic accuracy for each single biomarker and a combination of biomarkers for discrimination of all patient groups was calculated, and the results were displayed as a heatmap (FIG. 6A). Thereby, it was confirmed that the method using the combination of the three cytokines had the highest accuracy in differential diagnosis. In addition, a confusion matrix was drawn using the model to determine whether the diagnosis was accurately performed for a total of 50 patients. The diagnosis was accurately performed for 47 out of 50 patients, indicating a diagnostic accuracy of 94%. Specifically, one patient with non-infectious organ dysfunction was misdiagnosed as healthy control, one patient with infection was misdiagnosed as non-infectious organ dysfunction, and one patient with septic shock was misdiagnosed as infection. At this time, the accuracy, sensitivity, and specificity were calculated as 0.942, 0.940, and 0.985, respectively. Finally, the SOFA score, used in the existing diagnosis of sepsis, was integrated into the machine learning-based cytokine diagnosis method and the accuracy of the diagnosis was calculated through a confusion matrix, and as a result, it was shown that the analysis was performed accurately for 49 out of 50 people, indicating an accuracy of 98%. Compared to cytokine-based diagnosis, the accuracy increased by 4%, and the precision, sensitivity, and specificity also increased to 0.982, 0.980, and 0.995, respectively. The only case of diagnostic failure was misdiagnosis of infection as non-infectious organ dysfunction. This can be considered a very accurate result even when considering the case where clinical classification was incorrect due to false negatives because the blood sample did not contain bacteria or the bacteria had already died in the body and could not be cultured, when culturing the blood of an actual infected patient to confirm infection. These results show that the new diagnostic method based on the three cytokines is quite accurate on its own, but when linked with existing diagnostic methods, it enables highly accurate differential diagnosis of sepsis, suggesting that the diagnostic platform can make groundbreaking advances in diagnosis and treatment of sepsis in the future by determining the type of organ dysfunction and quantifying the severity of sepsis in actual field settings.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

1. A metal nanoparticle having a truncated octahedral structure composed of six (100) facets and eight (111) facets, wherein edges between four different adjacent (100) facets positioned with respect to any one of the (100) facets are truncated to form quadrangular connecting facets, wherein a concave channel is formed in each of the connecting facets.

2. The metal nanoparticle of claim 1, wherein the channel has a quadrangular groove shape having the edges as a center line.

3. The metal nanoparticle of claim 2, wherein the channel has a rectangular groove shape.

4. The metal nanoparticle of claim 1, wherein the (100) facets with truncated edges have a quadrangular shape.

5. The metal nanoparticle of claim 1, wherein the (111) facets with truncated edges have a triangular shape.

6. The metal nanoparticle of claim 1, wherein a ratio of an area of the (111) facet with truncated edges to an area of the (100) facet with truncated edges is 5.3 to 6.5.

7. The metal nanoparticle of claim 1, wherein the metal is any one selected from the group consisting of gold (Au), silver (Ag), copper (Cu), platinum (Pt), and palladium (Pd).

8. A plasmonic biosensor comprising:
a substrate; and
a metal nanoparticle array in which a plurality of metal nanoparticles according to claim 1 are arranged on the substrate.

9. The plasmonic biosensor of claim 8, wherein the metal nanoparticle array is configured so that the plurality of metal nanoparticles are in contact with each other.

10. The plasmonic biosensor of claim 8, wherein a capture antibody is bound to the metal nanoparticles.

11. The plasmonic biosensor of claim 8, further comprising a Raman probe comprising a gold nanoparticle and a detection antibody bound to the gold nanoparticle.

12. The plasmonic biosensor of claim 8, which is used to detect a sepsis biomarker.

13. The plasmonic biosensor of claim 12, wherein the sepsis biomarker is a myokine or an adipokine.

14. The plasmonic biosensor of claim 13, wherein the myokine is oncostatin M or leukemia inhibitory factor (LIF).

15. The plasmonic biosensor of claim 13, wherein the adipokine is visfatin.

16. A method of detecting a sepsis biomarker using the plasmonic biosensor according to claim 8.
